(19) United States
(12) Patent Application Publication
(10) Pub. No.: US 2024/0325412 A1
(43) Pub. Date: Oct. 3, 2024

(54) COMBINATIONS

(71) Applicant: Recurium IP Holdings, LLC, San Diego, CA (US)

(72) Inventors: Fernando Donate, Santa Monica, CA (US); Hooman Izadi, San Diego, CA (US); Jianhui Ma, San Diego, CA (US); Ahmed Abdi Ma, West Windsor, NJ (US); Kevin Duane Bunker, Escondido, CA (US); Peter Qinhua Huang, San Diego, CA (US)

(21) Appl. No.: 18/743,595

(22) Filed: Jun. 14, 2024

Related U.S. Application Data
(63) Continuation of application No. PCT/US2022/081600, filed on Dec. 14, 2022.
(60) Provisional application No. 63/375,510, filed on Sep. 13, 2022.
(60) Provisional application No. 63/265,444, filed on Dec. 15, 2021.

(51) Int. Cl. A61K 31/635; A61K 31/519; A61K 31/573; A61K 31/69; A61K 31/706; A61P 35/00

(52) U.S. Cl. CPC A61K 31/635; A61K 31/519; A61K 31/573; A61K 31/69; A61K 31/706; A61P 35/00

(57) ABSTRACT

Disclosed herein are combinations of compounds for treating a disease or condition, such as cancer.

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6, including U.S. Provisional Application Nos. 63/265,444, filed Dec. 15, 2021 and 63/375,510, filed Sep. 13, 2022, each of which is incorporated by reference in their entireties. The present application is a continuation of PCT Application No. PCT/US2022/081600, filed Dec. 14, 2022, which claims priority to U.S. Provisional Application Nos. 63/265,444, filed Dec. 15, 2021 and 63/375,510, filed Sep. 13, 2022, each of which is hereby incorporated by reference in their entireties.

### FIELD

The present application relates to the fields of chemistry, biochemistry and medicine. More particularly, disclosed herein are combination therapies, and methods of treating diseases and/or conditions with a combination therapies descried herein.

### DESCRIPTION

Cancers are a family of diseases that involve abnormal cell growth with the potential to invade or spread to other parts of the body. Cancer treatments today include surgery, hormone therapy, radiation, chemotherapy, immunotherapy, targeted therapy and combinations thereof. Survival rates vary by cancer type and by the stage at which the cancer is diagnosed. In 2019, roughly 1.8 million people will be diagnosed with cancer, and an estimated 606,880 people will die of cancer in the United States. Thus, there still exists a need for effective cancer treatments.

### SUMMARY

Some embodiments described herein relate to a combination of compounds that can include an effective amount of Compound (A), an effective amount of Compound (B) and an effective amount of Compound (C), or a pharmaceutically acceptable salt of any of the foregoing.

Some embodiments described herein relate to the use of a combination of compounds for treating a disease or condition, wherein the combination includes an effective amount of Compound (A), an effective amount of Compound (B) and an effective amount of Compound (C), or a pharmaceutically acceptable salt of any of the foregoing. Other embodiments described herein relate to the use of a combination of compounds in the manufacture of a medicament for treating a disease or condition, wherein the combination includes an effective amount of Compound (A), an effective amount of Compound (B) and an effective amount of Compound (C), or a pharmaceutically acceptable salt of any of the foregoing. Still other embodiments described herein relate to a combination of an effective amount of Compound (A), an effective amount of Compound (B) and an effective amount of Compound (C), or a pharmaceutically acceptable salt of any of the foregoing, for use in treating a disease or condition. Yet still other embodiments described herein relate to a method for treating a disease or condition that can include administering an effective amount of Compound (A), an effective amount of Compound (B) and an effective amount of Compound (C), or a pharmaceutically acceptable salt of any of the foregoing, in combination.

In some embodiments, the disease or condition can be a cancer described herein, such as triple negative breast cancer, multiple myeloma, acute myeloid leukemia and/or amyloidosis.

### DRAWINGS

FIG. 1 provides examples of Bcl-2 inhibitors.

FIG. 2 provides examples of Compound (C) agents.

FIG. 3 shows the effect of using a Compound (1A), or a pharmaceutically acceptable salt thereof, a Compound (B), or a pharmaceutically acceptable salt thereof, and Compound (C) alone or in combination on cell viability in OPM-2 cell line based on CTG assay.

FIG. 4 shows the effect of using a Compound (1A), or a pharmaceutically acceptable salt thereof, a Compound (B), or a pharmaceutically acceptable salt thereof, and Compound (C) alone or in combination on cell viability in MOLM13 and HL60 cell lines based on CTG assay.

FIG. 5 shows the effect of using a Compound (1A), or a pharmaceutically acceptable salt thereof, a Compound (B), or a pharmaceutically acceptable salt thereof, and Compound (C) alone or in combination on tumor volume in an HL-60 leukemia xenograft model.

FIG. 6 shows the effect of using a Compound (1A), or a pharmaceutically acceptable salt thereof, a Compound (B), or a pharmaceutically acceptable salt thereof, and Compound (C) alone or in combination on tumor volume in an OPM-2 multiple myeloma model.

FIG. 7 shows the effect of using a Compound (1A), or a pharmaceutically acceptable salt thereof, a Compound (B), or a pharmaceutically acceptable salt thereof, and Compound (C) alone or in combination on tumor volume in an KMS-12-BM multiple myeloma model.

FIG. 8 shows the effect of using a Compound (1A), or a pharmaceutically acceptable salt thereof, a Compound (B), or a pharmaceutically acceptable salt thereof, and Compound (C) alone or in combination on tumor volume in an MDA-MB-436 triple negative breast cancer model.

FIG. 9 shows the effect of using a Compound (1A), or a pharmaceutically acceptable salt thereof, and a Compound (B), or a pharmaceutically acceptable salt thereof, alone or in combination on tumor volume in a H23 non-small cell lung cancer model.

FIG. 10 shows the effect of using a Compound (1A), or a pharmaceutically acceptable salt thereof, and a Compound (B), or a pharmaceutically acceptable salt thereof, alone or in combination on tumor volume in a DMS53 small cell lung cancer model.

### DETAILED DESCRIPTION

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), a sulfuric acid, a nitric acid and a phosphoric acid (such as 2,3-dihydroxypropyl dihydrogen phosphate). Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, trifluoroacetic, benzoic, salicylic, 2-oxopentanedioic, or naphthalenesulfonic acid. Pharmaceutical salts can also be obtained by reacting a compound with a base to form a salt such as an ammonium salt, an alkali metal salt, such as a sodium, a potassium or a lithium salt, an alkaline earth metal salt, such as a calcium or a magnesium salt, a salt of a carbonate, a salt of a bicarbonate, a salt of organic bases such as dicyclohexylamine, N-methyl-D-glucamine, tris (hydroxymethyl) methylamine, C₁-C₇alkylamine, cyclohexylamine, triethanolamine, ethylenediamine, and salts with amino acids such as arginine and lysine. For compounds (A), (B) and (C), those skilled in the art understand that when a salt is formed by protonation of a nitrogen-based group (for example, NH₂), the nitrogen-based group can be associated with a positive charge (for example, NH₂ can become NH₃⁺) and the positive charge can be balanced by a negatively charged counterion (such as Cl⁻).

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastercomerically pure, diastereomerically enriched, or a stereoisomeric mixture. In addition, it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates, and hydrates. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Compounds

Some embodiments disclosed herein relate to the use of a combination of compounds for treating a disease or condition, wherein the combination can include an effective amount of Compound (A), an effective amount of Compound (B) and an effective amount of Compound (C), or a pharmaceutically acceptable salt of any of the foregoing. As provided herein Compound (A), or a pharmaceutically acceptable salt thereof, can be a Bcl-2 inhibitor, Compound (B), or a pharmaceutically acceptable salt thereof, can be a WEE1 inhibitor and Compound (C), or a pharmaceutically acceptable salt thereof, can be an agent, such as a chemotherapeutic agent.

Compound (A), including pharmaceutically acceptable salts thereof, can be

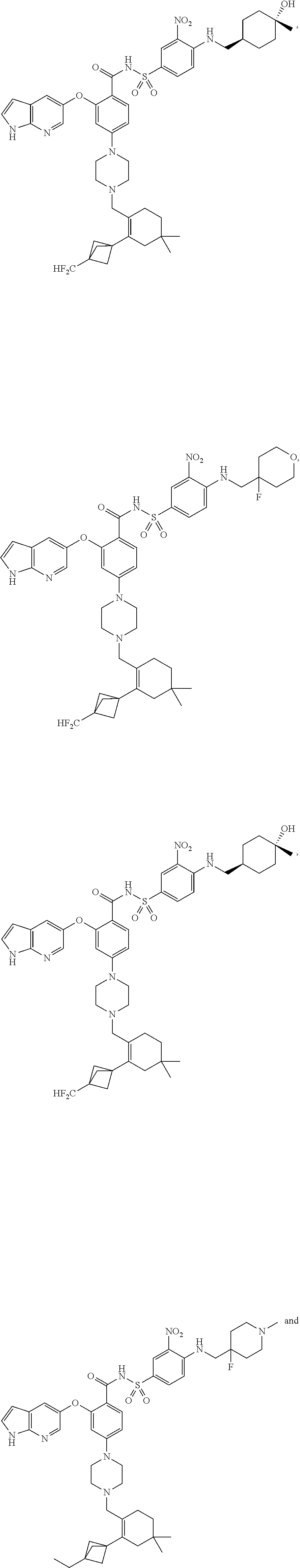

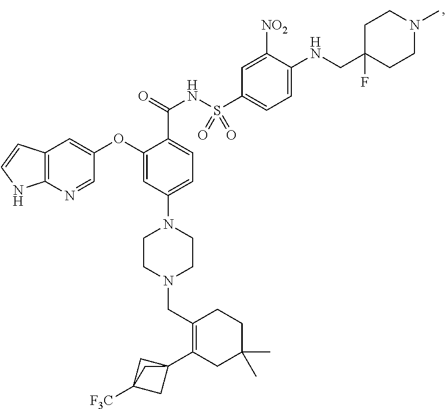 [SMILES: CN1CCC(F)(CNc2ccc(S(=O)(=O)NC(=O)c3ccc(N4CCN(CC5=C(C67CC(C(F)(F)F)(C6)C7)CC(C)(C)CC5)CC4)cc3Oc3cnc4[nH]ccc4c3)cc2[N+](=O)[O-])CC1]

selected from
including pharmaceutically acceptable salts thereof. Compound (B) can be

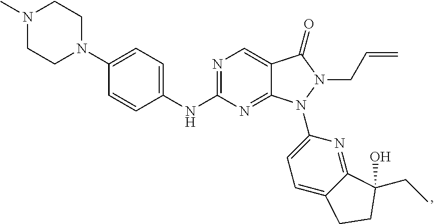 [SMILES: C=CCn1c(=O)c2cnc(Nc3ccc(N4CCN(C)CC4)cc3)nc2n1-c1ccc2c(n1)[C@@](O)(CC)CC2]

including pharmaceutically acceptable salts thereof. Compound (C), including pharmaceutically acceptable salts thereof, can be an agent selected from azacitidine, bendamustine, bortezomib, carfilzomib, ixazomib, busulfan, carboplatin, cytarabine, cyclophosphamide, cladribine, cisplatin, capecitabine, decitabine, dexamethasone, etoposide, fludarabine, gemcitabine, daunorubicin, doxorubicin, ifosfamide, methotrexate and vincristine, or a pharmaceutically acceptable salt of any of the foregoing.

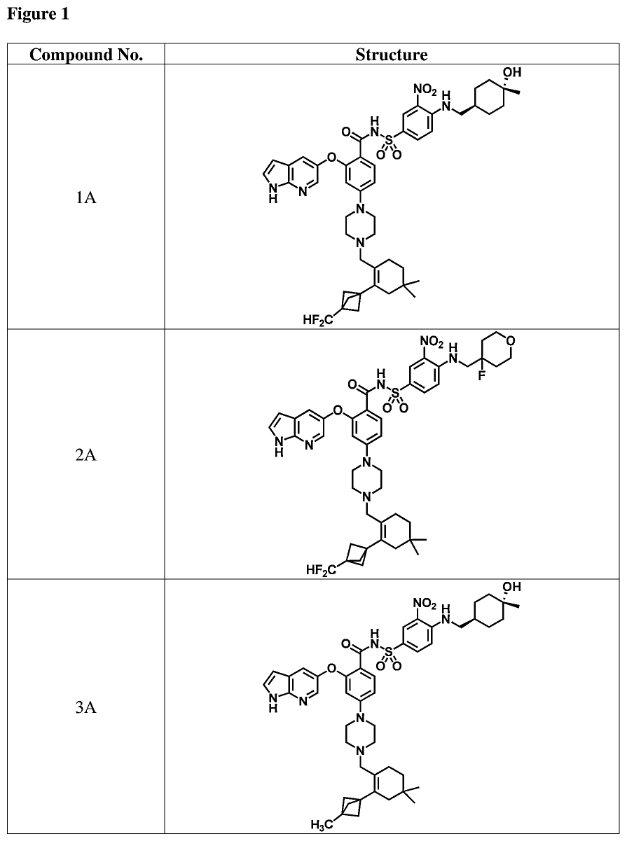

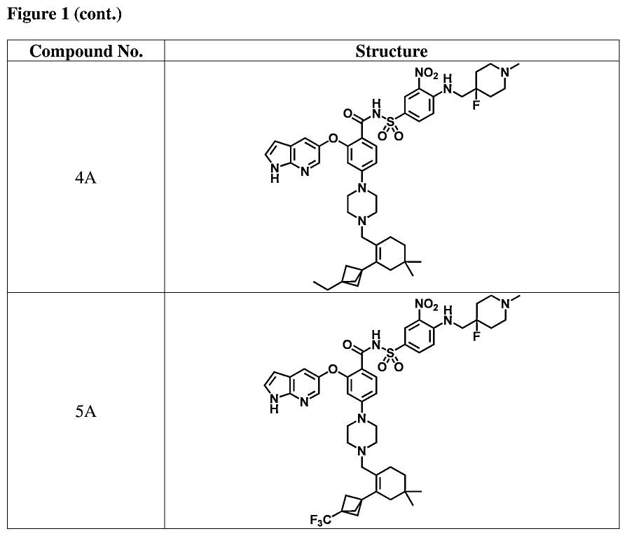

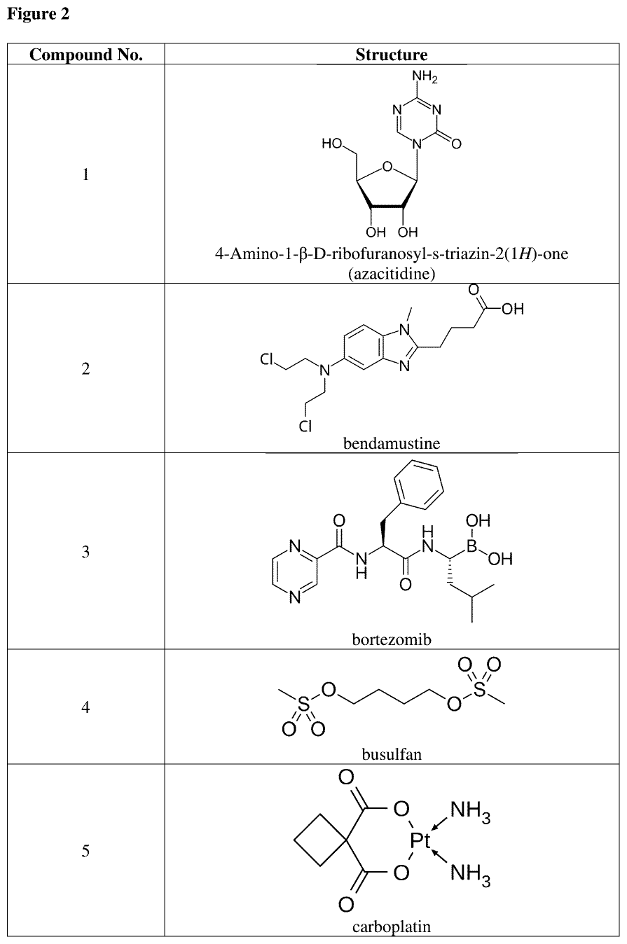

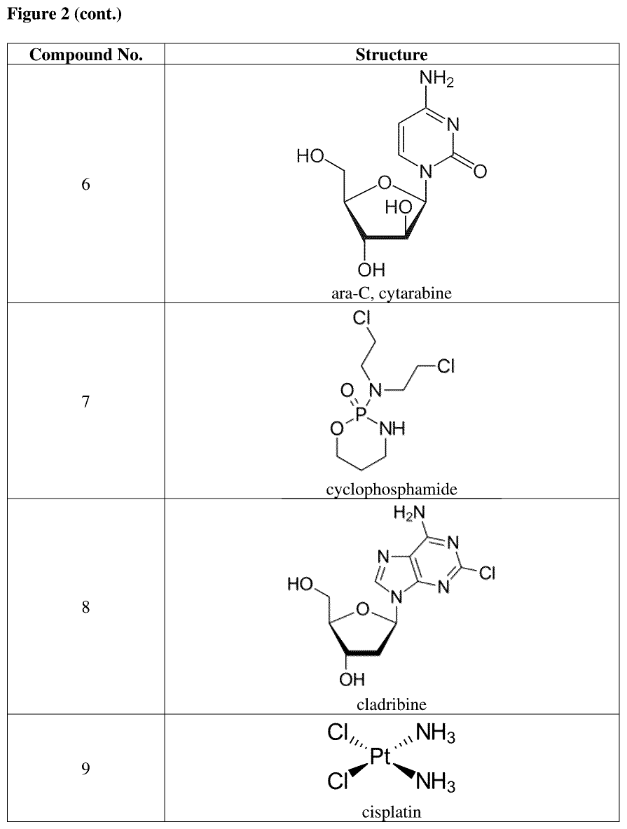

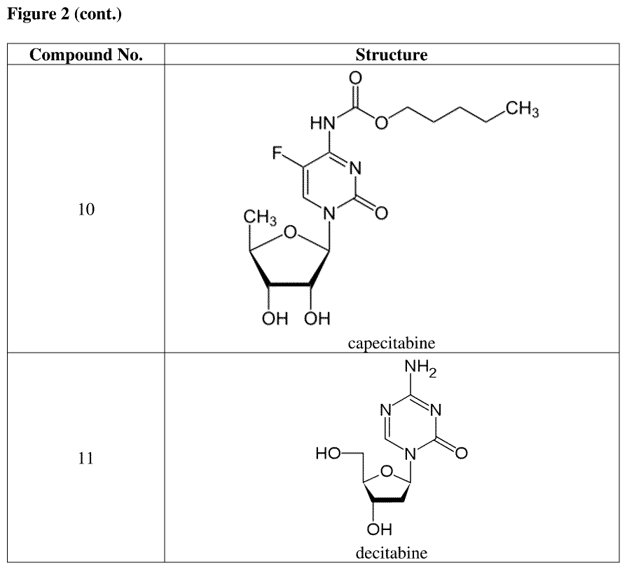

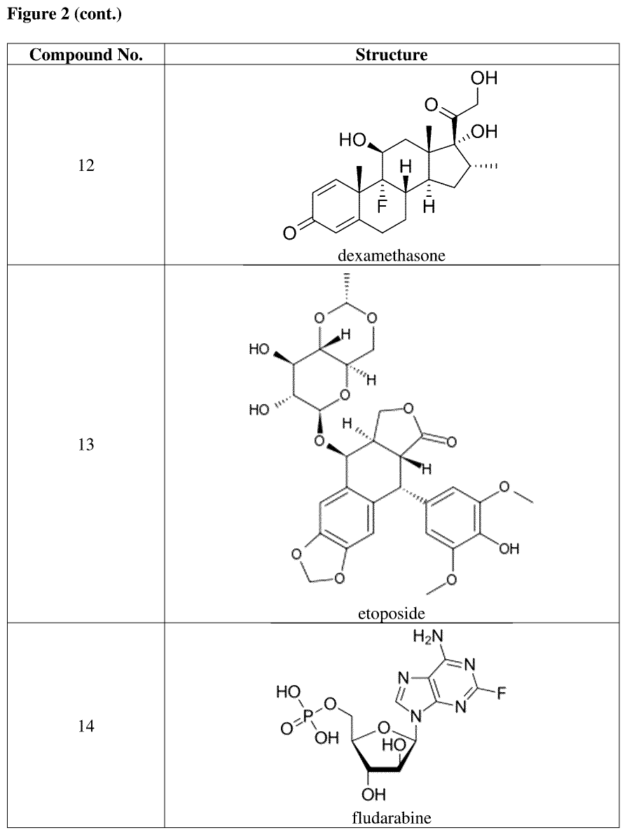

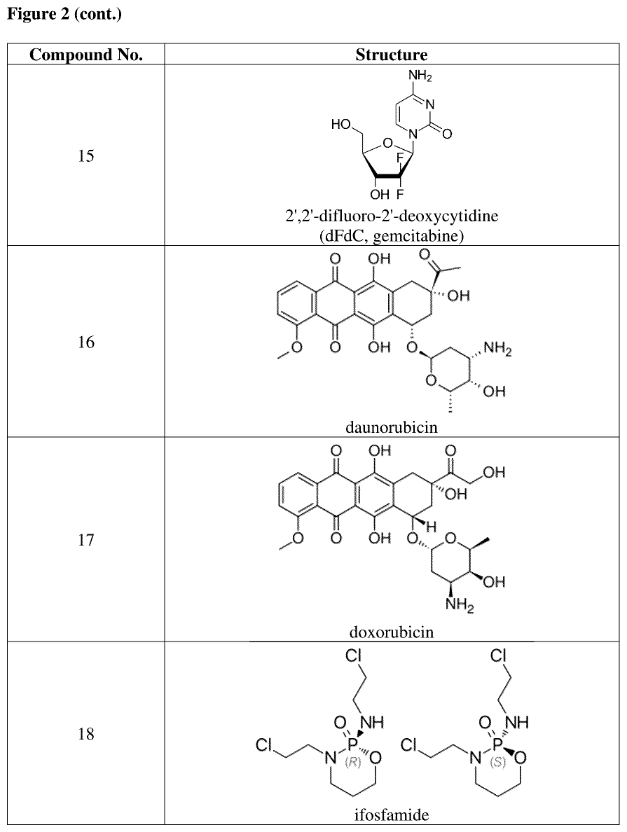

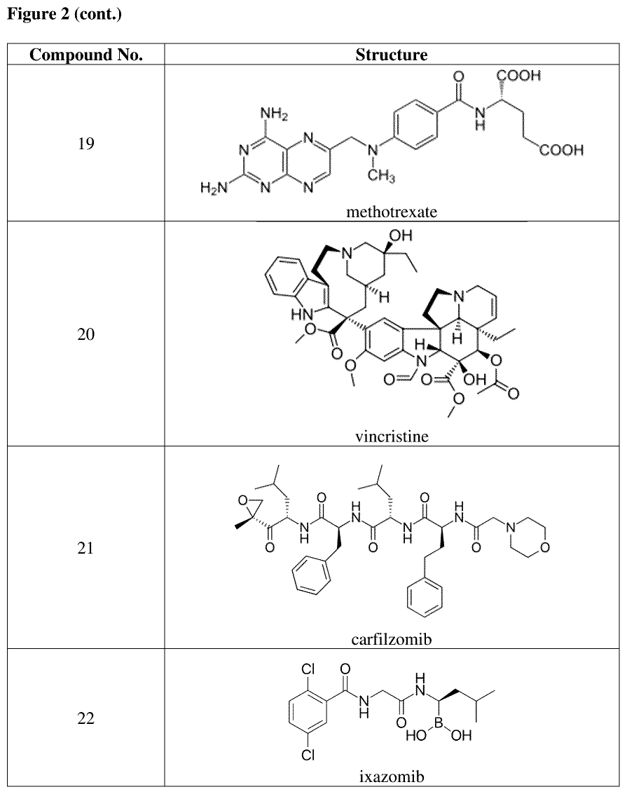

Embodiments of combinations of Compound (A), Compound (B) and Compound (C), including pharmaceutically acceptable salts of any of the foregoing, are provided in Table 1. In Table 1, numbers 1A-5A correspond to Compound (A) (including pharmaceutically acceptable salts thereof) provided in FIG. 1, “B” indicates Compound (B) (including pharmaceutically acceptable salts thereof) and the numbers 1-22 correspond to Compound (C) (including pharmaceutically acceptable salts thereof) provided in FIG. 2, including pharmaceutically acceptable salts thereof.

TABLE 1

| Cmpd:Cmpd | Cmpd:Cmpd | Cmpd:Cmpd | Cmpd:Cmpd |
|---|---|---|---|
| 1:1A:B | 5:1A:B | 9:1A:B | 13:1A:B |
| 2:1A:B | 6:1A:B | 10:1A:B | 14:1A:B |
| 3:1A:B | 7:1A:B | 11:1A:B | 15:1A:B |
| 4:1A:B | 8:1A:B | 12:1A:B | 16:1A:B |
| 17:1A:B | 19:2A:B | 21:3A:B | 1:5A:B |
| 18:1A:B | 20:2A:B | 22:3A:B | 2:5A:B |
| 19:1A:B | 21:2A:B | 1:4A:B | 3:5A:B |
| 20:1A:B | 22:2A:B | 2:4A:B | 4:5A:B |
| 21:1A:B | 1:3A:B | 3:4A:B | 5:5A:B |
| 22:1A:B | 2:3A:B | 4:4A:B | 6:5A:B |
| 1:2A:B | 3:3A:B | 5:4A:B | 7:5A:B |
| 2:2A:B | 4:3A:B | 6:4A:B | 8:5A:B |
| 3:2A:B | 5:3A:B | 7:4A:B | 9:5A:B |
| 4:2A:B | 6:3A:B | 8:4A:B | 10:5A:B |
| 5:2A:B | 7:3A:B | 9:4A:B | 11:5A:B |
| 6:2A:B | 8:3A:B | 10:4A:B | 12:5A:B |
| 7:2A:B | 9:3A:B | 11:4A:B | 13:5A:B |
| 8:2A:B | 10:3A:B | 12:4A:B | 14:5A:B |
| 9:2A:B | 11:3A:B | 13:4A:B | 15:5A:B |
| 10:2A:B | 12:3A:B | 14:4A:B | 16:5A:B |
| 11:2A:B | 13:3A:B | 15:4A:B | 17:5A:B |
| 12:2A:B | 14:3A:B | 16:4A:B | 18:5A:B |
| 13:2A:B | 15:3A:B | 17:4A:B | 19:5A:B |
| 14:2A:B | 16:3A:B | 18:4A:B | 20:5A:B |
| 15:2A:B | 17:3A:B | 19:4A:B | 21:5A:B |
| 16:2A:B | 18:3A:B | 20:4A:B | 22:5A:B |
| 17:2A:B | 19:3A:B | 21:4A:B | |
| 18:2A:B | 20:3A:B | 22:4A:B | |

The order of administration of compounds in a combination described herein can vary. In some embodiments, Compound (A), including pharmaceutically acceptable salts thereof, can be administered prior to Compound (B), or a pharmaceutically acceptable salt thereof, and Compound (C), along with pharmaceutically acceptable salts thereof. In other embodiments, Compound (A), including pharmaceutically acceptable salts thereof, can be administered prior to Compound (B), or a pharmaceutically acceptable salt thereof, and after administration of Compound (C), along with pharmaceutically acceptable salts thereof. In still other embodiments, Compound (A), including pharmaceutically acceptable salts thereof, can be administered prior to Compound (C), or a pharmaceutically acceptable salt thereof, and after administration of Compound (B), or a pharmaceutically acceptable salt thereof. In yet still other embodiments, Compound (A), including pharmaceutically acceptable salts thereof, can be administered after Compound (B), or a pharmaceutically acceptable salt thereof, and Compound (C), or a pharmaceutically acceptable salt thereof.

In some embodiments, Compound (B), including pharmaceutically acceptable salts thereof, can be administered prior to Compound (A), or a pharmaceutically acceptable salt thereof, and Compound (C), along with pharmaceutically acceptable salts thereof. In other embodiments, Compound (B), including pharmaceutically acceptable salts thereof, can be administered prior to Compound (A), along with pharmaceutically acceptable salts thereof, and after Compound (C), or a pharmaceutically acceptable salt thereof. In still other embodiments, Compound (B), including pharmaceutically acceptable salts thereof, can be administered prior to Compound (C), or a pharmaceutically acceptable salt thereof, and after Compound (A), including pharmaceutically acceptable salts thereof. In yet still other embodiments, Compound (B), or pharmaceutically acceptable salt thereof, can be administered after Compound (A), along with pharmaceutically acceptable salts thereof, and Compound (C), or a pharmaceutically acceptable salt thereof.

In some embodiments, Compound (C), including pharmaceutically acceptable salts thereof, can be administered prior to Compound (A), or a pharmaceutically acceptable salt thereof, and Compound (B), along with pharmaceutically acceptable salts thereof. In other embodiments, Compound (C), including pharmaceutically acceptable salts thereof, can be administered prior to Compound (A), or a pharmaceutically acceptable salt thereof, and after Compound (B), or a pharmaceutically acceptable salt thereof. In still other embodiments, Compound (C), including pharmaceutically acceptable salts thereof, can be administered prior to Compound (B), including pharmaceutically acceptable salts thereof, and after Compound (A), along with pharmaceutically acceptable salts thereof. In yet still other embodiments, Compound (C), or pharmaceutically acceptable salt thereof, can be administered after Compound (A), along with pharmaceutically acceptable salts thereof, and Compound (B), or a pharmaceutically acceptable salt thereof.

In some embodiments, Compound (A), including pharmaceutically acceptable salts thereof, can be administered concomitantly with Compound (B), or a pharmaceutically acceptable salt thereof, and/or Compound (C), or a pharmaceutically acceptable salt thereof. In other embodiments, Compound (B), including pharmaceutically acceptable salts thereof, can be administered concomitantly with Compound (A), or a pharmaceutically acceptable salt thereof, and/or Compound (C), or a pharmaceutically acceptable salt thereof.

There may be several advantages for using a combination of compounds described herein. For example, combining compounds that attack multiple pathways at the same time, can be more effective in treating a cancer, such as those described herein, compared to when the compounds of combination are used as monotherapy.

In some embodiments, a combination as described herein (for example, Compound (A), Compound (B) and Compound (C), along with pharmaceutically acceptable salts of any of the foregoing) can decrease the number and/or severity of side effects that can be attributed to a compound described herein, such as Compound (B), or a pharmaceutically acceptable salt thereof. In other embodiments, a combination as described herein (such as Compound (A), Compound (B) and Compound (C), along with pharmaceutically acceptable salts of any of the foregoing) can decrease the number and/or severity of side effects that can be attributed to Compound (B), or a pharmaceutically acceptable salt thereof.

Using a combination of compounds described herein can results in additive, synergistic or strongly synergistic effect. A combination of compounds described herein can result in an effect that is not antagonistic.

In some embodiments, a combination as described herein of Compound (A), including pharmaceutically acceptable salts thereof, Compound (B), along with pharmaceutically acceptable salts thereof, and Compound (C), including pharmaceutically acceptable salts thereof, can result in an additive effect. In other embodiments, a combination as described herein (such as Compound (A), Compound (B) and Compound (C), along with pharmaceutically acceptable salts of any of the foregoing) can result in a synergistic effect. In still other embodiments, a combination as described herein (for example, Compound (A), Compound (B) and Compound (C), along with pharmaceutically acceptable salts of any of the foregoing) can result in a strongly synergistic effect. In yet still other embodiments, a combination as described herein (for example, Compound (A), Compound (B) and Compound (C), along with pharmaceutically acceptable salts of any of the foregoing) is not antagonistic.

As used herein, the term “antagonistic” means that the activity of the combination of compounds is less compared to the sum of the activities of the compounds in combination when the activity of each compound is determined individually (i.e., as a single compound). As used herein, the term “synergistic effect” means that the activity of the combination of compounds is greater than the sum of the individual activities of the compounds in the combination when the activity of each compound is determined individually. As used herein, the term “additive effect” means that the activity of the combination of compounds is about equal to the sum of the individual activities of the compounds in the combination when the activity of each compound is determined individually.

A potential advantage of utilizing a combination as described herein may be a reduction in the required amount(s) of the compound(s) that is effective in treating a disease condition disclosed herein (such as triple negative breast cancer, multiple myeloma, acute myeloid leukemia and/or amyloidosis) compared to when each compound is administered as a monotherapy. For example, the amount of Compound (B), or a pharmaceutically acceptable salt thereof, used in a combination described herein can be less compared to the amount of Compound (B), or a pharmaceutically acceptable salt thereof, needed to achieve the same reduction in a disease marker (for example, tumor size) when administered as a monotherapy. Another potential advantage of utilizing a combination as described herein is that the use of two or more compounds having different mechanisms of action can create a higher barrier to the development of resistance compared to when a compound is administered as monotherapy. Additional advantages of utilizing a combination as described herein may include little to no cross resistance between the compounds of a combination described herein; different routes for elimination of the compounds of a combination described herein; and/or little to no overlapping toxicities between the compounds of a combination described herein.

### Pharmaceutical Compositions

Compound (A), including pharmaceutically acceptable salts thereof, can be provided in a pharmaceutical composition. Compound (B), including pharmaceutically acceptable salts thereof, can be provided in a pharmaceutical composition. Similarly, Compound (C), including pharmaceutically acceptable salts thereof, can be provided in a pharmaceutical composition. Examples of Compound (A), Compound (B) and Compound (C) are described herein.

The term “pharmaceutical composition” refers to a mixture of one or more compounds and/or salts disclosed herein with other chemical components, such as diluents, carriers and/or excipients. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, and salicylic acid. Pharmaceutical compositions will generally be tailored to the specific intended route of administration.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks appreciable pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the pH and isotonicity of human blood.

As used herein, an “excipient” refers to an essentially inert substance that is added to a pharmaceutical composition to provide, without limitation, bulk, consistency, stability, binding ability, lubrication, disintegrating ability etc., to the composition. For example, stabilizers such as antioxidants and metal-chelating agents are excipients. In an embodiment, the pharmaceutical composition comprises an antioxidant and/or a metal-chelating agent. A “diluent” is a type of excipient.

In some embodiments, Compounds (B), along with pharmaceutically acceptable salts thereof, can be provided in a pharmaceutical composition that includes Compound (A), including pharmaceutically acceptable salts thereof, and/or Compound (C), including pharmaceutically acceptable salts thereof. In some embodiments, Compounds (C), along with pharmaceutically acceptable salts thereof, can be provided in a pharmaceutical composition that includes Compound (A), including pharmaceutically acceptable salts thereof, and/or Compound (B), including pharmaceutically acceptable salts thereof. In other embodiments, Compound (B), along with pharmaceutically acceptable salts thereof, can be administered in a pharmaceutical composition that is separate from a pharmaceutical composition that includes Compound (A), including pharmaceutically acceptable salts thereof. In still other embodiments, Compounds (B), along with pharmaceutically acceptable salts thereof, can be administered in a pharmaceutical composition that is separate from a pharmaceutical composition that includes Compound (C), including pharmaceutically acceptable salts thereof. In still other embodiments, Compounds (C), or a pharmaceutically acceptable salt thereof, can be administered in a pharmaceutical composition that is separate from a pharmaceutical composition that includes Compound (A), along with pharmaceutically acceptable salts thereof. In some embodiments, Compounds (A), or a pharmaceutically acceptable salt thereof, can be administered in a pharmaceutical composition that is separate from a pharmaceutical composition that includes Compound (B), or a pharmaceutically acceptable salt thereof. In other embodiments, Compounds (A), or a pharmaceutically acceptable salt thereof, can be administered in a pharmaceutical composition that is separate from a pharmaceutical composition that includes Compound (B), or a pharmaceutically acceptable salt thereof, and separate from a pharmaceutical composition that includes Compound (C), or a pharmaceutically acceptable salt thereof.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound, salt and/or composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In some embodiments, Compound (A), including pharmaceutically acceptable salts thereof, can be administered orally. In some embodiments, Compound (B), including pharmaceutically acceptable salts thereof, can be administered intravenously, orally, intramuscularly, subcutaneous and/or topically. In some embodiments, Compound (C), including pharmaceutically acceptable salts thereof, can be administered orally. In some embodiments, Compound (A), including pharmaceutically acceptable salts thereof, can be provided to a subject by the same route of administration as Compound (B), along with pharmaceutically acceptable salts thereof. In other embodiments, Compound (A), including pharmaceutically acceptable salts thereof, can be provided to a subject by a different route of administration as Compound (B), along with pharmaceutically acceptable salts thereof. In still other embodiments, Compound (C), including pharmaceutically acceptable salts thereof, can be provided to a subject by the same route of administration as Compound (B), along with pharmaceutically acceptable salts thereof. In yet still other embodiments, Compound (C), including pharmaceutically acceptable salts thereof, can be provided to a subject by a different route of administration as Compound (B), along with pharmaceutically acceptable salts thereof.

One may also administer the compound, salt and/or composition in a local rather than systemic manner, for example, via injection or implantation of the compound directly into the affected area, often in a depot or sustained release formulation. Furthermore, one may administer the compound in a targeted drug delivery system, for example, in a liposome coated with a tissue-specific antibody. The liposomes will be targeted to and taken up selectively by the organ. For example, intranasal or pulmonary delivery to target a respiratory disease or condition may be desirable.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical carrier may also be prepared, placed in an appropriate container, and labeled for treatment of an indicated condition.

### Uses and Methods of Treatment

As provided herein, in some embodiments, a combination of compounds described herein, such as a combination that includes an effective amount of Compound (A), an effective amount of one or more of Compound (B), and an effective amount of one or more of Compound (C), or a pharmaceutically acceptable salt of any of the foregoing, can be used to treat a disease or condition (including a disease or condition described herein, for example, triple negative breast cancer, multiple myeloma, acute myeloid leukemia and/or amyloidosis) in a subject in need thereof.

Some embodiments disclosed herein relate to the use of a combination of compounds for treating triple negative breast cancer, wherein the combination can include an effective amount of Compound (A), and an effective amount of Compound (B), or a pharmaceutically acceptable salt of any of the foregoing, wherein Compound (A) can be

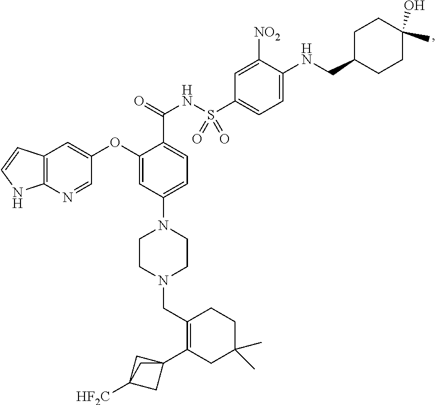

or a pharmaceutically acceptable salt thereof.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the subject can be human. In some embodiments, the subject can be a child and/or an infant, for example, a child or infant with a fever. In other embodiments, the subject can be an adult.

As used herein, the terms “treat,” “treating,” “treatment,” “therapeutic,” and “therapy” do not necessarily mean total cure or abolition of the disease or condition. Any alleviation of any undesired signs or symptoms of the disease or condition, to any extent can be considered treatment and/or therapy. Furthermore, treatment may include acts that may worsen the subject's overall feeling of well-being or appearance.

The term “effective amount” is used to indicate an amount of an active compound, or pharmaceutical agent, that elicits the biological or medicinal response indicated. For example, an effective amount of compound, salt or composition can be the amount needed to prevent, alleviate or ameliorate symptoms of the disease or condition, or prolong the survival of the subject being treated. This response may occur in a tissue, system, animal or human and includes alleviation of the signs or symptoms of the disease or condition being treated. Determination of an effective amount is well within the capability of those skilled in the art, in view of the disclosure provided herein. The effective amount of the compounds disclosed herein required as a dose will depend on the route of administration, the type of animal, including human, being treated and the physical characteristics of the specific animal under consideration. The dose can be tailored to achieve a desired effect, but will depend on such factors as weight, diet, concurrent medication and other factors which those skilled in the medical arts will recognize.

For example, an effective amount of a compound, or radiation, is the amount that results in: (a) the reduction, alleviation or disappearance of one or more symptoms caused by the cancer, (b) the reduction of tumor size, (c) the elimination of the tumor, and/or (d) long-term disease stabilization (growth arrest) of the tumor.

In some embodiments, the disease or condition can be triple negative breast cancer. In some embodiments, the triple negative breast cancer can be local triple negative breast cancer (as used herein, “local” breast cancer means the cancer has not spread to other areas of the body). In other embodiments, the triple negative breast cancer can be metastatic triple negative breast cancer. A subject can have a breast cancer that has not been previously treated.

In some embodiments, the triple negative breast cancer can be present in subject, wherein the subject can be a woman. As women approach middle-age, a woman can be in a stage of menopause. In some embodiments, the subject can be a premenopausal woman. In other embodiments, the subject can be a perimenopausal woman. In still other embodiments, the subject can be a menopausal woman. In yet still other embodiments, the subject can be a postmenopausal woman. In other embodiments, the triple negative breast cancer can be present in a subject, wherein the subject can be a man. The serum estradiol level of the subject can vary.

In some embodiments, the serum estradiol level (E2) of the subject can be in the range of >15 pg/mL to 350 pg/mL. In other embodiments, the serum estradiol level (E2) of the subject can be ≤15 pg/mL. In other embodiments, the serum estradiol level (E2) of the subject can be ≤10 pg/mL.

In some embodiments, the disease or condition can be amyloidosis. Amyloidosis refers to a group of diseases caused by protein misfolding and aggregation into highly ordered amyloid fibrils that deposit in tissues. Some of the types of amyloidosis include amyloid light-chain (AL) amyloidosis, amyloid type A (AA) amyloidosis, dialysis-related amyloidosis (DRA), familial or hereditary amyloidosis, age-related (senile) systemic amyloidosis, and organ-specific amyloidosis. If left untreated, amyloidosis may result in progressive organ damage. Current amyloidosis treatments include chemotherapy, stem cell transplant therapy, steroid therapy, treatment of the underlying disorder, and combinations thereof. In some embodiments, combination therapies described herein can be used to treat amyloidosis. In some embodiments, the amyloidosis can be selected from amyloid light-chain (AL) amyloidosis, amyloid type A (AA) amyloidosis, dialysis-related amyloidosis (DRA), familial or hereditary amyloidosis, age-related (senile) systemic amyloidosis, organ-specific amyloidosis and combinations thereof. A subject can have an amyloidosis that has not been previously treated.

In some cases, following treatment, a subject can relapse or have reoccurrence of the disease or condition. As used herein, the terms “relapse” and “reoccurrence” are used in their normal sense as understood by those skilled in the art. For example, the breast cancer can be recurrent breast cancer. In some embodiments, the subject has relapsed after a previous treatment. As an example, a subject who had been previously treated for triple negative breast cancer with an agent described herein can have a reoccurrence of triple negative breast cancer. A non-limiting list of exemplary agents that a subject who has been diagnosed with recurrent triple negative breast cancer was previously treated with includes chemotherapeutic agents, PARP inhibitors and/or PD-L1 inhibitors, such as those described herein. As an additional example, amyloidosis can be a recurrent amyloidosis. In some embodiments, the subject has relapsed after a previous treatment for AL amyloidosis.

In some embodiments, the disease or condition can be multiple myeloma. In some embodiments, the disease or condition can be acute myeloid leukemia (AML). As with other diseases and conditions described herein, a subject can have relapsed after being previously treated. For example, a subject who is diagnosed with multiple myeloma may have been previously treated with an anti-CD38 monoclonal antibody, a proteasome inhibitor, and/or an immunomodulatory agent. In some embodiments, a subject who is diagnosed with acute myeloid leukemia may have been previously treated with a chemotherapeutic agent.

The amount of compound, salt and/or composition required for use in treatment will vary not only with the particular compound or salt selected but also with the route of administration, the nature and/or symptoms of the disease or condition being treated and the age and condition of the patient and will be ultimately at the discretion of the attendant physician or clinician. In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the dosage ranges described herein in order to effectively and aggressively treat particularly aggressive diseases or conditions.

As will be readily apparent to one skilled in the art, the useful in vivo dosage to be administered and the particular mode of administration will vary depending upon the age, weight, the severity of the affliction, the mammalian species treated, the particular compounds employed and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies. For example, useful dosages of a compounds (A), (B) and/or (C), or pharmaceutically acceptable salts of any of the foregoing, can be determined by comparing their in vitro activity, and in vivo activity in animal models. Such comparison can be done by comparison against an established drug, such as cisplatin and/or gemcitabine)

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the disease or condition to be treated and to the route of administration. The severity of the disease or condition may, for example, be evaluated, in part, by standard prognostic evaluation methods. Further, the dose and perhaps dose frequency, will also vary according to the age, body weight and response of the individual patient. A program comparable to that discussed above may be used in veterinary medicine.

Compounds, salts and compositions disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### EXAMPLES

Additional embodiments are disclosed in further detail in the following examples, which are not in any way intended to limit the scope of the claims.

Cell proliferation was measured using the CellTiter-Glo® Luminescent Cell Viability Assay. The assay involved the addition of a single reagent (CellTiter-Glo® Reagent) directly to cells cultured in serum-supplemented medium. OPM-2, MOLM-13 and HL-60 cells were cultured according to ATCC recommendations and were seeded at 10,000 cells per well.

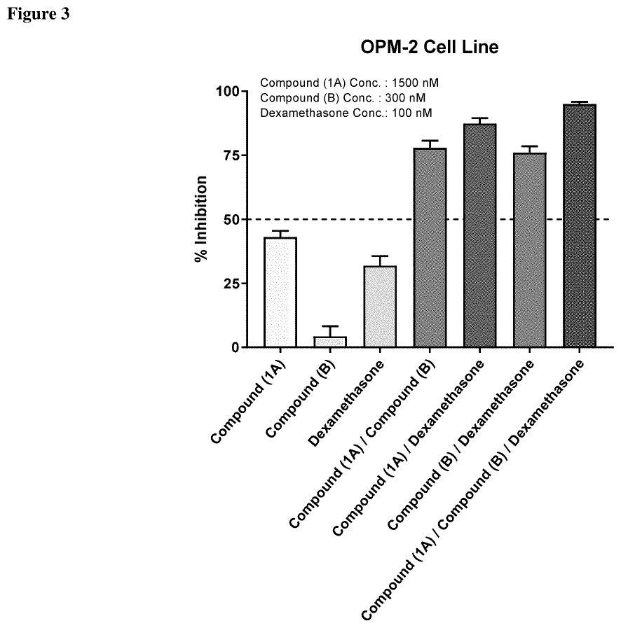

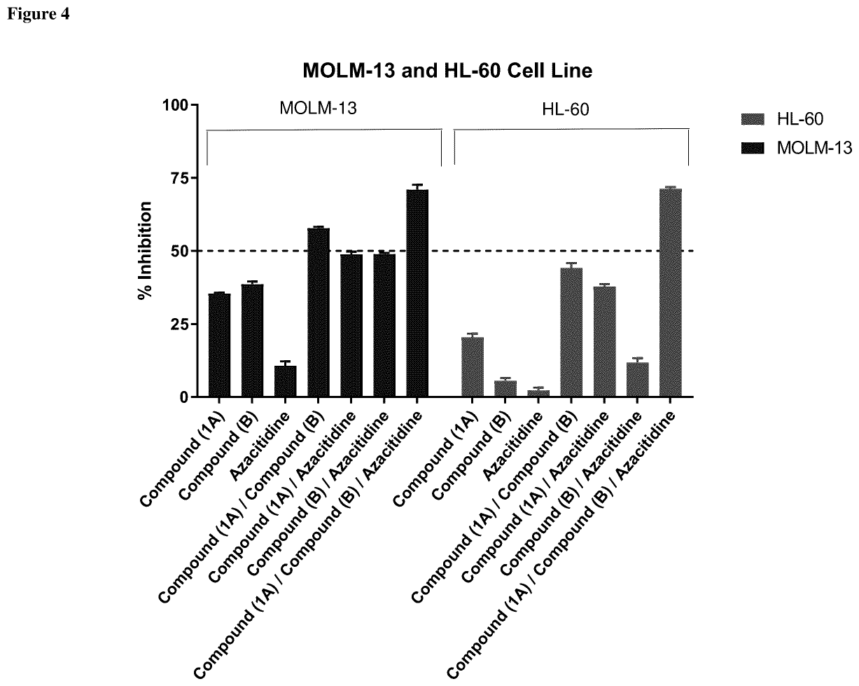

Compound (1 A), Compound (B) and Dexamethasone or Azacitidine were prepared as a DMSO stock solution (10 mM). For OPM-2 cell line, compounds were tested in triplicate using Dexamethasone; MOLM-13 and HL-60 cell lines compound were tested in triplicate using Azacitidine. The respective concentrations are shown in FIGS. 3 and 4 and summarized in Tables 2 and 3. Plates were incubated at 37° C., 5% CO₂ for 72 h and then equilibrated at room temperature (RT) for approximately 30 min. An equal-volume amount of CellTiter-Glo® Reagent (100 μL) was added to each well. Plates were mixed for 2 min on an orbital shaker to induce cell lysis and then incubated at RT for 10 min to stabilize the luminescent signal. Luminescence (RLU (relative light unit)) was recorded using a SpectraMAX, M5e plate reader according to CellTiter-Glo protocol. Percent inhibition was calculated using the following formula: % inhibition=(RLU*100/(RLU of the cell background)).

TABLE 2

| | OPM-2 | |
|---|---|---|
| | Concentration | Inhibition |
| Compound/Combination | (nM) | (%) |
| Compound (1A) | 1500 | 43 |
| Compound (B) | 300 | 4.3 |
| Dexamethasone | 100 | 32 |
| Compound (1A) + Compound (B) | 1500 + 300 | 78 |
| Compound (1A) + Dexamethasone | 1500 + 100 | 87 |
| Compound (B) + Dexamethasone | 300 + 100 | 76 |
| Compound (1A) + Compound (B) + | 1500 + 300 + 100 | 95 |
| Dexamethasone | | |

TABLE 3

| | MOLM-13 | | HL-60 | |
|---|---|---|---|---|
| | Concentration | Inhibition | Concentration | Inhibition |
| Compound/Combination | (nM) | (%) | (nM) | (%) |
| Compound (1A) | 4 | 35.4 | 40 | 20.4 |
| Compound (B) | 12 | 38.6 | 120 | 5.6 |
| Azacitidine | 300 | 10.7 | 1500 | 2.3 |
| Compound (1A) + | 4 + 120 | 57.8 | 40 + 120 | 44.2 |
| Compound (B) | | | | |
| Compound (1A) + Azacitidine | 4 + 300 | 48.8 | 40 + 1500 | 37.8 |
| Compound (B) + Azacitidine | 120 + 300 | 48.9 | 120 + 1500 | 11.8 |
| Compound (1A) + | 4 + 120 + 300 | 71 | 40 + 120 + 1500 | 71.3 |
| Compound (B) + Azacitidine | | | | |

### Xenograft Tumor Model

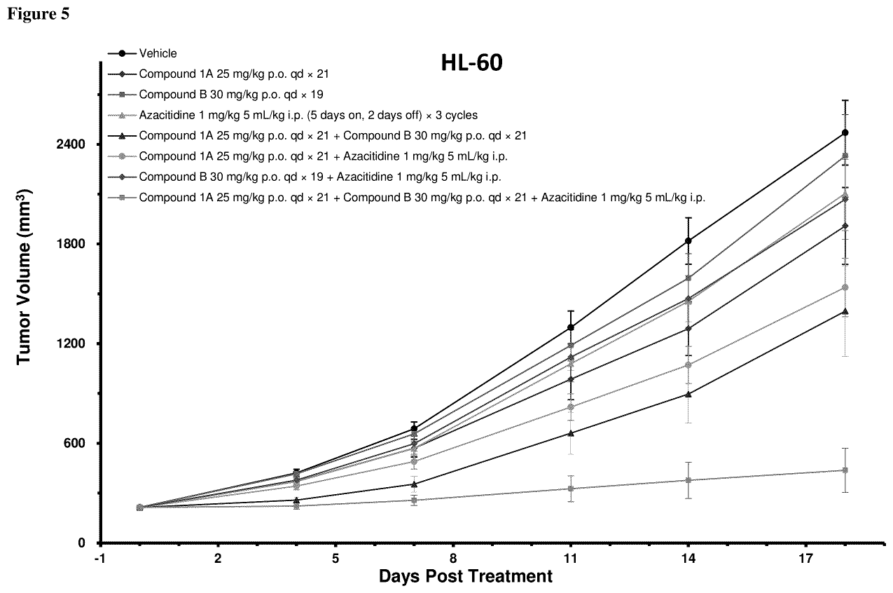

Mice were inoculated with HL-60 cells subcutaneously on the right flank with the single cell suspension of 95% viable tumor cells (1×10⁷) in 100 μL IMDM Matrigel mixture (1:1 ratio) without serum for the tumor development. The treatment was started when the mean tumor size reached approximately 214 mm³, with individual tumor size ranging from 185-245 mm³. Animals were randomly distributed into treatment groups of 10 animals each and dosed with vehicle and indicated compounds at indicated dosage and frequency shown in FIG. 5 and Table 4. In FIG. 5, the bottom line is Compound (1A) 25 mg/kg p.o. qd×21+Compound (B) 30 mg/kg p.o. pd×21+azacitidine 1 mg/kg IP 5 days on 2 days off×3 cycle, the second from the bottom line is Compound (1A)+Compound (B), the third from the bottom line is Compound (1A)+azacytidine, the fourth from the bottom line is Compound (1A), the top line is Vehicle, the second from the top line is Compound (B), and the remaining two lines are azacytidine (indicated with triangles) and Compound (B)+azacytidine (indicated with diamonds). Tumor volumes were evaluated twice per week to calculate tumor volume over time, and mice were weighed twice per week as a surrogate for signs of toxicity. Tumor growth inhibition (TGI) was calculated using the following equation TGI=(1−(Td−T0)/(Cd−C0))×100%. Td and Cd are the mean tumor volumes of the treated and control animals, and T0 and C0 are the mean tumor volumes of the treated and control animals at the start of the experiment. The tumor regression was defined as individual tumor volume (TV) decrease (terminal TV compared to initial TV). FIG. 5 and Table 4 illustrate that single agent, double agent and triple treatment of Compound (1A) at 25 mg/kg, Compound (B) at 30 mg/kg and azacitidine at 1 mg/kg. The combination of Compound (1A) (25 mg/kg)+Compound (B) (30 mg/kg) and azacitidine exhibited 90% tumor growth inhibition at day 18.

TABLE 4

| | TGI % |
|---|---|
| Compound/Combination | (DAY 18) |
| Compound (1A) 25 mg/kg | 25 |
| Compound (B) 30 mg/kg | 6 |
| Azacitidine 1 mg/kg | 16 |
| Compound (1A) + Compound (B) | 48 |
| Compound (1A) + Azacitidine | 41 |
| Compound (B) + Azacitidine | 18 |
| Compound (1A) + Compound (B) + Azacitidine | 90 |

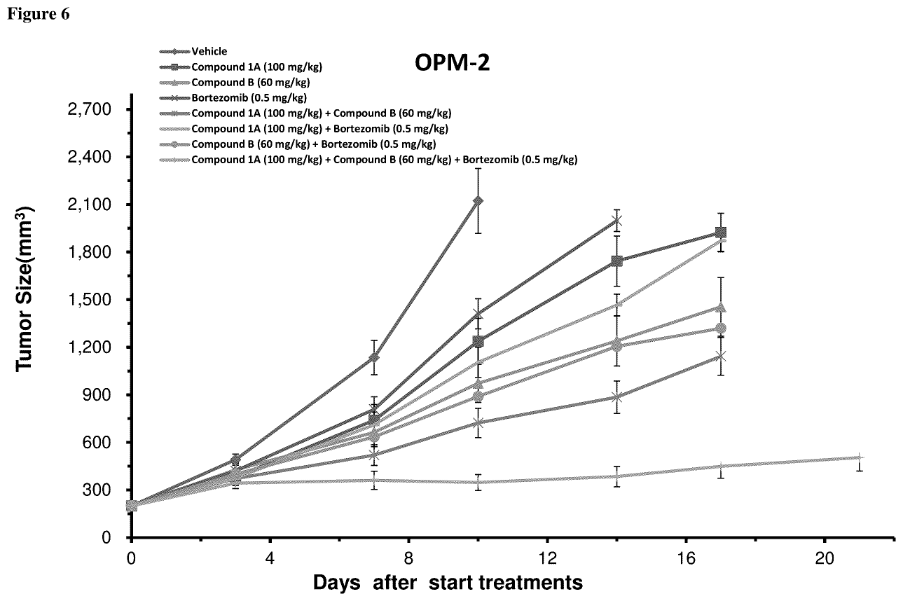

Mice were inoculated with OPM-2 cells subcutaneously on the right flank with the single cell suspension of 95% viable tumor cells (1×10⁷) in 100 μL IMDM Matrigel mixture (1:1 ratio) without serum for the tumor development. The treatment was started when the mean tumor size reached approximately 200 mm³, with individual tumor size ranging from 185-225 mm³. Animals were randomly distributed into treatment groups of 10 animals each and dosed with vehicle and indicated compounds at indicated dosage and frequency shown in FIG. 6 and Table 5. In FIG. 6, the bottom line is Compound (1A) 100 mg/kg p.o. qd×21+Compound (B) 60 mg/kg p.o. pd×21+Bortezomib 0.5 mg/kg IP BIW (twice a week)×3, the second from the bottom line is Compound (1A)+Compound (B), the third from the bottom line is Compound (B)+Bortezomib, the fourth from the bottom line is Compound (B), the top line is Vehicle, the second from the top is Bortezomib, the third from the top line (indicted with squares) is Compound (1A) and the fourth from the top line is Compound (1A)+Bortezomib. Tumor volumes were evaluated twice per week to calculate tumor volume over time, and mice were weighed twice per week as a surrogate for signs of toxicity. Tumor growth inhibition (TGI) was calculated using the following equation TGI=(1−(Td−T0)/(Cd−C0))×100%. Td and Cd are the mean tumor volumes of the treated and control animals, and T0 and C0 are the mean tumor volumes of the treated and control animals at the start of the experiment. The tumor regression was defined as individual tumor volume (TV) decrease (terminal TV compared to initial TV). FIG. 6 and Table 5 illustrate that single agent, double agent, and triple treatment of Compound (1A) at 100 mg/kg, Compound (B) at 60 mg/kg and bortezomib at 0.5 mg/kg. The combination of Compound (1A) (100 mg/kg)+Compound (B) (60 mg/kg) and bortezomib exhibited 87% tumor growth inhibition at day 17. Additionally, triple combination resulted in tumor stagnation compared to single and double combinations.

TABLE 5

| | TGI % |
|---|---|
| Compound/Combination | (DAY 17) |
| Compound (1A) 25 mg/kg | 18 |
| Compound (B) 30 mg/kg | 33 |
| Bortezomib 0.5 mg/kg | 0 |
| Compound (1A) + Compound (B) | 68 |
| Compound (1A) + Bortezomib | 41 |
| Compound (B) + Bortezomib | 53 |
| Compound (1A) + Compound (B) + Bortezomib | 87 |

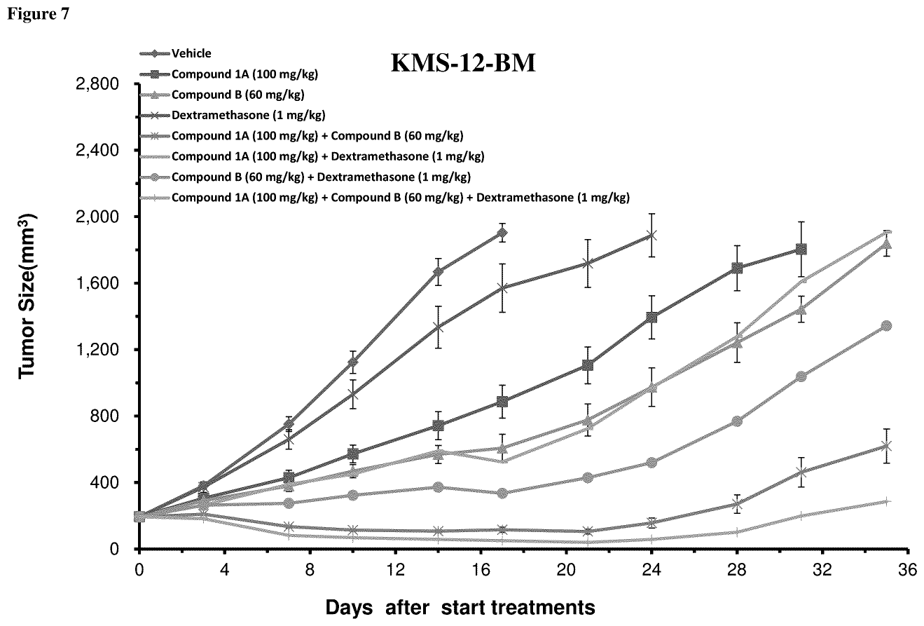

Mice were inoculated with KMS-12-BM cells subcutaneously on the right flank with the single cell suspension of 95% viable tumor cells (1×10⁷) in 100 μL IMDM Matrigel mixture (1:1 ratio) without serum for the tumor development. The treatment was started when the mean tumor size reached approximately 193 mm³, with individual tumor size ranging from 185-220 mm³. Animals were randomly distributed into treatment groups of 10 animals each and dosed with vehicle and indicated compounds at indicated dosage and frequency shown in FIG. 7 and Table 6. In FIG. 7, the bottom line is Compound (1A) 100 mg/kg p.o. qd×21+Compound (B) 60 mg/kg p.o. pd×21+Dexamethasone 1 mg/kg IP 5 days on 2 days off ×3, the second from the bottom line is Compound (1A)+Compound (B), the third from the bottom line is Compound (B)+Dexamethasone, the fourth from the bottom line (indicated with triangles) is Compound (B), the top line is Vehicle, the second from the top line is Dexamethasone, the third from the top line is Compound (1A) and the remaining line is Compound (1A)+Dexamethasone. Tumor volumes were evaluated twice per week to calculate tumor volume over time, and mice were weighed twice per week as a surrogate for signs of toxicity. Tumor growth inhibition (TGI) was calculated using the following equation TGI=(1−(Td−T0)/(Cd−C0))×100%. Td and Cd are the mean tumor volumes of the treated and control animals, and T0 and C0 are the mean tumor volumes of the treated and control animals at the start of the experiment. The tumor regression was defined as individual tumor volume (TV) decrease (terminal TV compared to initial TV). The percent tumor regression was calculated using the formula: (1−(Td/T0))×100%. FIG. 7 and Table 6 illustrate that single agent, double agent, and triple treatment of Compound (1A) at 100 mg/kg, Compound (B) at 60 mg/kg and Dexamethasone at 1 mg/kg. The combination of Compound (1A) (100 mg/kg)+Compound (B) (60 mg/kg) and Dexamethasone exhibited 113% tumor growth inhibition at day 10 and 79% tumor regression on day 21.

TABLE 6

| | | TUMOR | TUMOR |
|---|---|---|---|
| | TGI % | REGRESSION % | REGRESSION % |
| Compound/Combination | (DAY 14) | (DAY 21) | (DAY 28) |
| Compound (1A) 25 mg/kg | 62.8 | — | — |
| Compound (B) 30 mg/kg | 74.5 | — | — |
| Dexamethasone 1 mg/kg | 22.5 | — | — |
| Compound (1A) + Compound (B) | 105.9 | 45 | — |
| Compound (1A) + | 73.1 | — | — |
| Dexamethasone | | | |
| Compound (B) + Dexamethasone | 87.9 | — | — |
| Compound (1A) + Compound (B) + | 109.2 | 79 | 48.2 |
| Dexamethasone | | | |

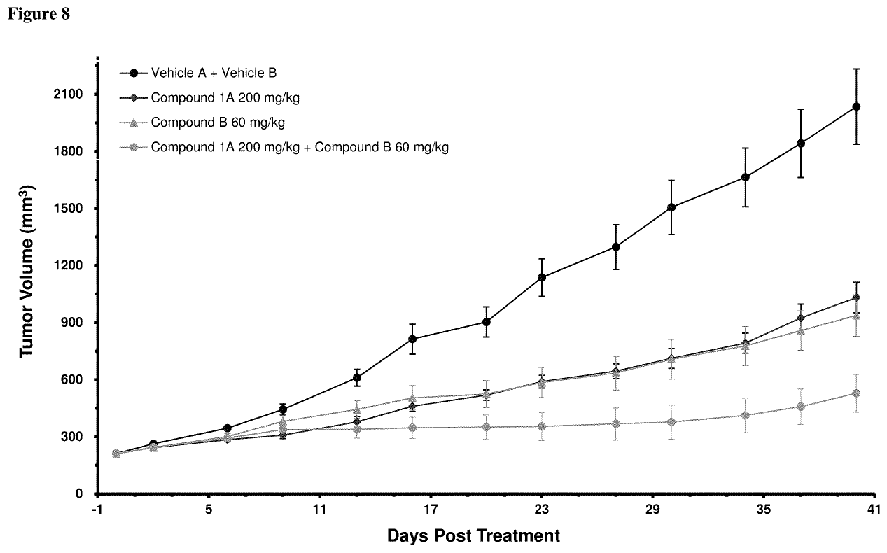

Mice were inoculated with MDA-MB-436 cells (TNBC breast cancer cell line) subcutaneously on the right flank with the single cell suspension of 95% viable tumor cells (1×10⁷) in 100 μL DMDM Matrigel mixture (1:1 ratio) without serum for the tumor development. The treatment was started when the mean tumor size reached approximately 210 mm³ for each model (with individual tumor volumes between 180-240 mm³). Animals were randomly distributed into treatment groups of 8 animals each and dosed with vehicle and indicated compounds at indicated dosage and frequency shown in FIG. 8 and Table 7. In FIG. 8, the top line (indicated with circles) is Vehicle control, the second from the top line (indicated with diamonds) is Compound (1A) (200 mg/kg 10 mL/kg p.o. qd×40) and the third line from the top (indicated with triangles) Compound (B) (60 mg/kg 10 mL/kg p.o. qd×40). The bottom line (indicated with circles) is Compound (1A) (200 mg/kg 10 mL/kg p.o. qd×40)+Compound (B) (60 mg/kg 10 mL/kg p.o. qd×40). Tumor volumes were evaluated twice per week to calculate tumor volume over time, and mice were weighed twice per week as a surrogate for signs of toxicity. Tumor growth inhibition (TGI) was calculated using the following equation TGI=(1−(Td−T0)/(Cd−C0))×100%. Td and Cd are the mean tumor volumes of the treated and control animals, and T0 and C0 are the mean tumor volumes of the treated and control animals at the start of the experiment. The tumor regression was defined as individual tumor volume (TV) decrease (terminal TV compared to initial TV). FIG. 8 and Table 7 illustrate that single agent treatment of Compound (1A) at 200 mg/kg resulted in in 60% efficacy and single agent treatment with Compound (B) at 60 mg/kg resulted in 55% efficacy. In contrast, the combination of Compound (1A) (200 mg/kg) and Compound (B) (60 mg/kg) exhibited significant TGI of 83% on day 40.

TABLE 7

| | DOSE | |
|---|---|---|
| Compound | (mg/kg) | TGI % |
| Vehicle | — | — |
| Compound (B) | 60 | 55 |
| Compound (1A) | 200 | 60 |
| Compound (B) + Compound (1A) | 60 + 200 | 83 |

### Multiple Myeloma Models

Multiple myeloma (MM) cell lines are selected that contain at (11;14) translocation (i.e., KMS-12BM) or do not contain at (11;14) translocation (i.e., OPM-2). It is understood that compound activity in MM models is predictive of activity in amyloidosis (e.g., AL amyloidosis) models and/or patients.

The combination effect of Compound (A) with dexamethasone are studied in a KMS-12-BM mouse model. Mice are inoculated with KMS-12-BM cells subcutaneously on the right flank with the single cell suspension of 95% viable tumor cells (1×10⁷) in 200 μL RPMI-1640 Matrigel mixture (1:1 ratio) without serum for the tumor development. The treatment is started when the mean tumor size reached approximately 200 mm³, with individual tumor size ranging from 180-220 mm³. Animals are randomly distributed into treatment groups of 10 animals each and each grouping are dosed with vehicle or compound(s) at a predetermined dosages and frequencies. Tumor volumes are evaluated twice per week to calculate tumor volume over time, and mice are weighed twice per week as a surrogate for signs of toxicity. Tumor growth inhibition (TGI) is calculated using the following equation TGI=(1−(T_{d}−T₀)/(C_{d}−C₀))×100%. T_{d} and C_{d} are the mean tumor volumes of the treated and control animals, and T₀ and C₀ are the mean tumor volumes of the treated and control animals at the start of the experiment.

The combination effect of Compound (A) with bortezomide is studied in a KMS-12-BM mouse model. Mice are inoculated with KMS-12-BM cells subcutaneously on the right flank with the single cell suspension of 95% viable tumor cells (1×10⁷) in 200 μL RPMI-1640 Matrigel mixture (1:1 ratio) without serum for the tumor development. The treatment is started when the mean tumor size reached approximately 200 mm³, with individual tumor size ranging from 180-220 mm³. Animals are randomly distributed into treatment groups of 10 animals each and each grouping was dosed with vehicle or indicated compound(s) at a predetermined dosages and frequencies. Tumor volumes are evaluated twice per week to calculate tumor volume over time, and mice are weighed twice per week as a surrogate for signs of toxicity. Tumor growth inhibition (TGI) is calculated using the following equation TGI=(1−(T_{d}−T₀)/(C_{d}−C₀))×100%. T_{d} and C_{d} are the mean tumor volumes of the treated and control animals, and T₀ and C₀ are the mean tumor volumes of the treated and control animals at the start of the experiment.

### Lung Cancer Models

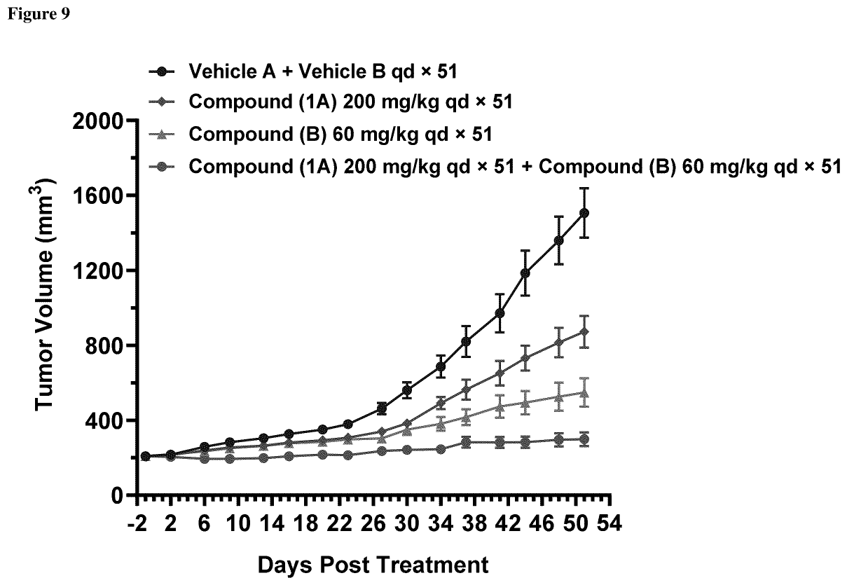

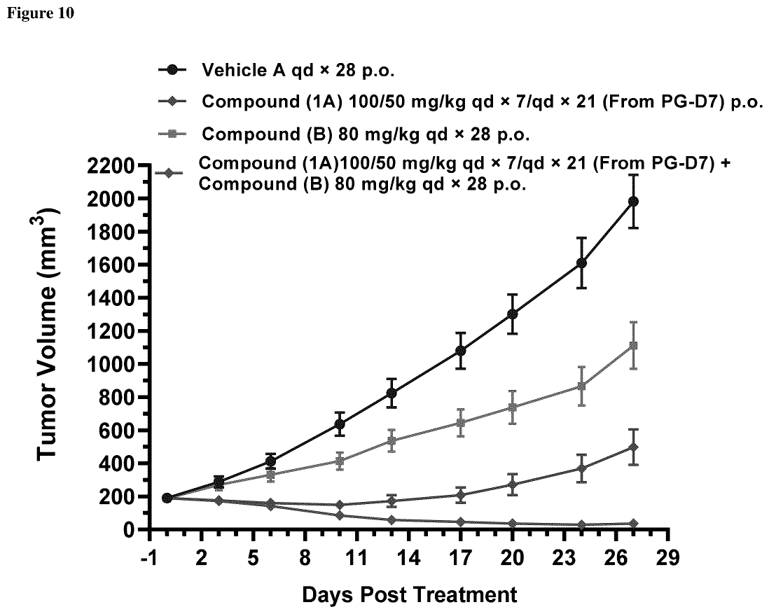

Data was generated using the Xenograft Tumor Model provided in WO 2021/127044 with H23 (non-small cell lung) or DMS53 (small cell lung cells) cells in place of HL-60 cells utilizing a combination of Compound (1A) and Compound (B). The data demonstrates that a dual combination of Compound (1A)+Compound (B) has increased efficacy compared to the efficacy when each aforementioned compound is used as a single agent. The results are shown in FIGS. 9 and 10. As shown in both figures, the efficacy of the dual combination of Compound (1A) and Compound (B) was maintained using even different dosing schedules. In FIG. 9, the top line (indicated with circles is vehicle), the second from top line (indicated with diamonds) is Compound (1A), the second from the bottom line (indicated with (triangles) is Compound (B), and the bottom line (indicated with circles) is Compound (1A)+Compound (B). In FIG. 10, the top line (indicated with circles is vehicle), the second from the top line (indicated with squares) is Compound (B), the second from the bottom line (indicated with diamonds) is Compound (1A), and the bottom line (indicated with diamonds) is Compound (1A)+Compound (B).

The data in Tables 2-6 and FIGS. 3-7 demonstrate that triple combinations of Compound (A)—a Bcl-2 inhibitor, Compound (B)—a WEE1 inhibitor and an anti-cancer drug (dexamethasone, bortezomib, and azacitidine) are more efficacious for treating a cancer compared to single and double agents, such as multiple myeloma (KMS-12-BM and OPM-2) and leukemia (MOLM13 and HL60). In addition, as shown in Table 7 and FIGS. 9 and 10, dual combinations of Compound (A)—a Bcl-2 inhibitor, and Compound (B)—a WEE1 inhibitor, are effective against cancer, for example, non-small cell and small cell lung cancer.

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the disclosure.

What is claimed is:

1. Use of a combination of compounds for treating a disease or condition, wherein the combination includes an effective amount of Compound (A), an effective amount of Compound (B) and an effective amount of Compound (C), or a pharmaceutically acceptable salt of any of the foregoing, wherein:
Compound (A) is a Bcl-2 inhibitor selected from the group consisting of: 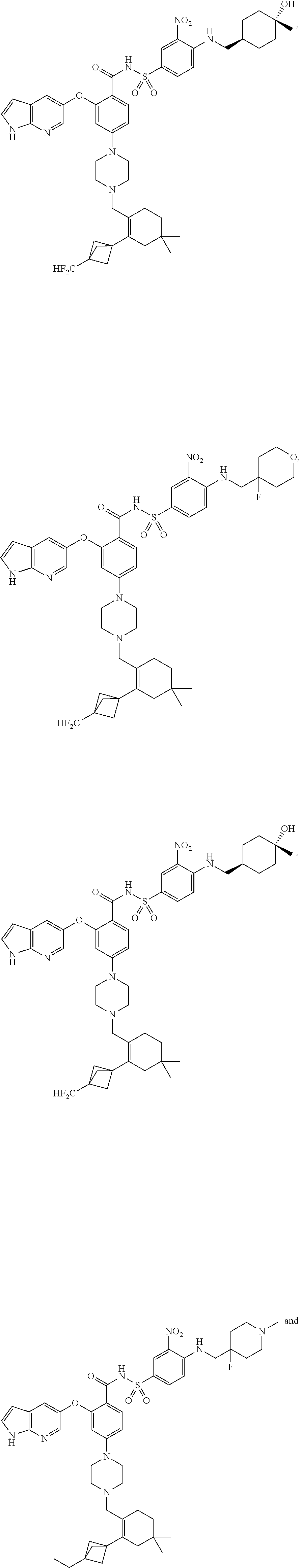 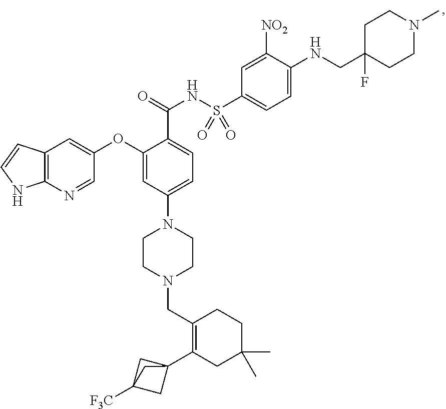 [SMILES: CN1CCC(F)(CNc2ccc(S(=O)(=O)NC(=O)c3ccc(N4CCN(CC5=C(C67CC(C(F)(F)F)(C6)C7)CC(C)(C)CC5)CC4)cc3Oc3cnc4[nH]ccc4c3)cc2[N+](=O)[O-])CC1]
or a pharmaceutically acceptable salt thereof;
Compound (B) is 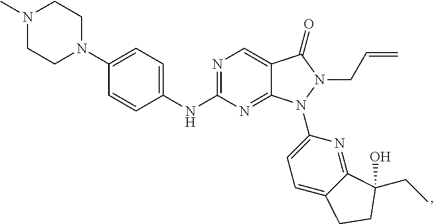 [SMILES: C=CCn1c(=O)c2cnc(Nc3ccc(N4CCN(C)CC4)cc3)nc2n1-c1ccc2c(n1)[C@@](O)(CC)CC2]
or a pharmaceutically acceptable salt thereof; and
Compound (C) is an agent selected from the group consisting of azacitidine, bendamustine, bortezomib, carfilzomib, ixazomib, busulfan, carboplatin, cytarabine, cyclophosphamide, cladribine, cisplatin, capecitabine, decitabine, dexamethasone, etoposide, fludarabine, gemcitabine, daunorubicin, doxorubicin, ifosfamide, methotrexate and vincristine, or a pharmaceutically acceptable salt of any of the foregoing.

2. The use of claim 1, wherein the BCL-2 inhibitor is 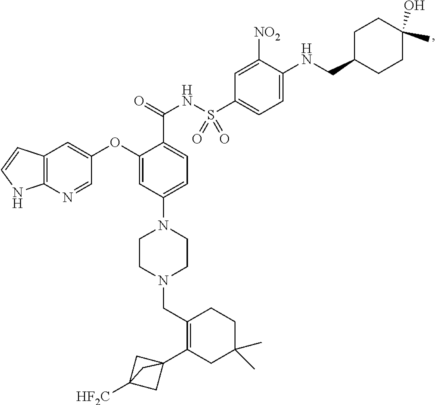
or a pharmaceutically acceptable salt thereof.

3. The use of claim 1, wherein the BCL-2 inhibitor is 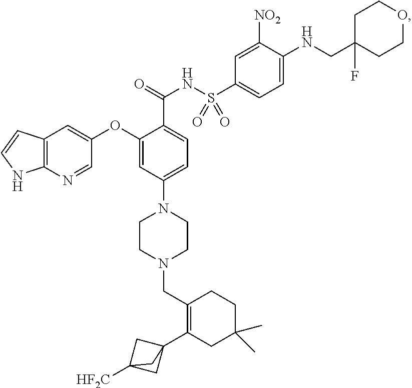
or a pharmaceutically acceptable salt thereof.

4. The use of claim 1, wherein the BCL-2 inhibitor is 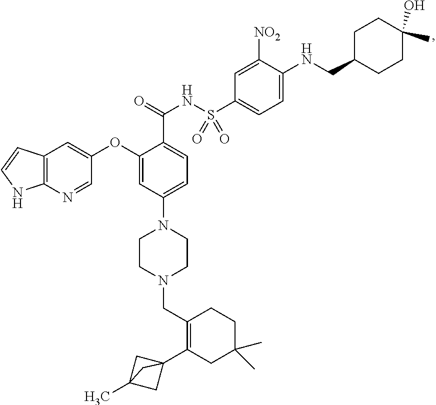 [SMILES: CC1(C)CCC(CN2CCN(c3ccc(C(=O)NS(=O)(=O)c4ccc(NC[C@H]5CC[C@](C)(O)CC5)c([N+](=O)[O-])c4)c(Oc4cnc5[nH]ccc5c4)c3)CC2)=C(C23CC(C)(C2)C3)C1 ; ","]
or a pharmaceutically acceptable salt thereof.

5. The use of claim 1, wherein the BCL-2 inhibitor is 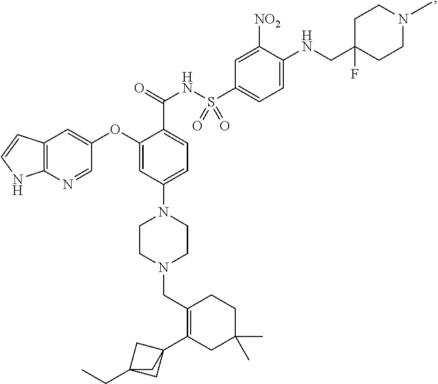 [SMILES: "," ; CCC12CC(C3=C(CN4CCN(c5ccc(C(=O)NS(=O)(=O)c6ccc(NCC7(F)CCN(C)CC7)c([N+](=O)[O-])c6)c(Oc6cnc7[nH]ccc7c6)c5)CC4)CCC(C)(C)C3)(C1)C2]
or a pharmaceutically acceptable salt thereof.

6. The use of claim 1, where in BCL-2 inhibitor is 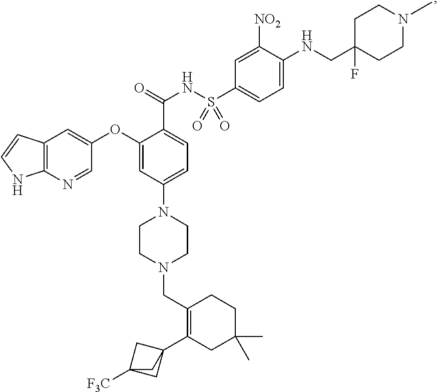 [SMILES: "," ; CN1CCC(F)(CNc2ccc(S(=O)(=O)NC(=O)c3ccc(N4CCN(CC5=C(C67CC(C(F)(F)F)(C6)C7)CC(C)(C)CC5)CC4)cc3Oc3cnc4[nH]ccc4c3)cc2[N+](=O)[O-])CC1]
or a pharmaceutically acceptable salt thereof.

7. The use of any one of claims 1-6, wherein Compound (C) is azacitidine.

8. The use of any one of claims 1-6, wherein Compound (C) is bortezomib.

9. The use of any one of claims 1-6, wherein Compound (C) is dexamethasone.

10. The use of any one of claims 1-9, wherein the disease or condition is selected from the group consisting of triple negative breast cancer, multiple myeloma, acute myeloid leukemia, lung cancer and amyloidosis.

11. The use of claim 10, wherein the disease or condition is triple negative breast cancer.

12. The use of claim 11, wherein the triple negative breast cancer is local breast cancer.

13. The use of claim 11, wherein the triple negative breast cancer is metastatic breast cancer.

14. The use of claim 11, wherein the triple negative breast cancer is recurrent breast cancer.

15. The use of any one of claims 11-14, wherein the triple negative breast cancer has been previously treated with one or more agents selected from the group consisting of a chemotherapeutic agent, a PARP inhibitor and a PD-L1 inhibitor.

16. The use of any one of claim 11-15, wherein the triple negative breast cancer is present in a woman.

17. The use of claim 16, wherein the subject is a premenopausal woman.

18. The use of claim 16, wherein the subject is a perimenopausal woman.

19. The use of claim 16, wherein the subject is a menopausal woman.

20. The use of claim 16, wherein the triple negative breast cancer is present in a postmenopausal woman.

21. The use of any one of claim 11-15, wherein the triple negative breast cancer is present a man.

22. The use of any one of claim 11-21, wherein the triple negative breast cancer is present in a subject that has a serum estradiol level in the range of >15 pg/mL to 350 pg/mL.

23. The use of any one of claim 11-21, wherein the triple negative breast cancer is present in a subject that has a serum estradiol level ≤15 pg/mL.

24. The use of any one of claim 11-21, wherein the triple negative breast cancer is present in a subject that has a serum estradiol level ≤10 pg/mL.

25. The use of any one of claims 1-10, wherein the disease or condition is amyloidosis.

26. The use of any one of claims 1-10, wherein the disease or condition is multiple myeloma.

27. The use of any one of claims 1-10, wherein the disease or condition is acute myeloid leukemia.

28. The use of any one of claims 1-10, wherein the disease or condition is lung cancer.

29. The use of claim 28, wherein the lung cancer is non-small cell lung cancer.

30. The use of claim 28, wherein the lung cancer is small cell lung cancer.

31. The use of any one of claim 1-13 or 16-30, wherein the disease or disorder has not been previously treated.
